(19) United States
(12) Patent Application Publication
(10) Pub. No.: US 2010/0268286 A1
(43) Pub. Date: Oct. 21, 2010

(54) METHOD FOR SECURING A MEDICAL ELEMENT IN AN IMPLANT, AND MEDICAL ELEMENT

(75) Inventors: Michael Augthun, Mulheim (DE); Thomas Boschemeyer, Herdecke (DE); Volkhard-Hagen Clostermann, Hagen (DE); Klaus Haselhuhn, Aachen (DE); Nico Dragotto, Lunen (DE); Manfred Peters, Wolfenbuttel (DE); Hubertus Spiekermann, Haan (DE); Karl-Wilhelm Theis, Leichlingen (DE)

Correspondence Address: Friedrich Von Rohrscheidt, 7400 Whitepine Road, Richmond, VA, 23237, US

(73) Assignee: HERSEUS LULZER GMBH, HANAU (DE)

(21) Appl. No.: 11/917,318

(22) PCT Filed: Jun. 12, 2006
(86) PCT No.: PCT/EP2006/005595

§ 371 Date: Aug. 5, 2009

(30) Foreign Application Priority Data
Jun. 13, 2005 (DE) 10 2005 027 401.3

(51) Int. Cl. A61B 17/56; A61C 13/10

(52) U.S. Cl. 606/86 R; 433/191

(57) ABSTRACT

The invention relates to medical element and a method for securing the medical element in an implant, where the medical element has a connecting pin which is received in a receiving recess of the implant, the pin having a projection on at least one wing of a clip element and movable transversally relative to the longitudinal extension and brought into engagement with a radially outwardly facing corresponding depression in a jacket surface of the receiving recess such that the wing is subsequently secured against radial movement by hardening of a curable compound in the receiving recess.

The invention relates on the one hand to a method for securing a medical element in an implant, in which method a connecting pin of the medical element is first received in a receiving recess of the implant extending in the longitudinal direction into an interior of the implant, and a projection on at least one wing of a clip element of the connecting pin, movable transversally with respect to the longitudinal direction, is brought into engagement with a radially outwardly facing depression in a jacket surface of the receiving recess, and on the other hand to a medical element with a connecting pin which can be received in a receiving recess of an implant implantable in a receiving bore in a bone, with the receiving recess extending in the longitudinal direction into an interior of the implant, and the connecting pin having a clip element with at least one wing movable transversely with respect to the longitudinal direction and, on said wing, a radially outwardly protruding projection which can be brought into engagement with a corresponding depression in a jacket surface of the receiving recess.

Within the scope of implant systems, medical elements can be fastened to the bone in an especially careful manner with such methods: A sleeve-like implant with a simple cover cap is inserted at first into the bone. As soon as this implant, which at first is without function and is therefore not loaded mechanically, has healed in and is rigidly connected with the bone, any desired medical element in the dental area such as a substitute tooth, an impression post or a gingival former can be inserted. The medical element can be joined quickly and in a simple manner with the receiving recess of the implant as a result of a clip connection and said connection can also be detached again very easily and quickly.

Individual missing natural teeth can be replaced by artificial, but fixed substitute teeth. Such implant systems are also suitable for closing larger gaps in teeth with several natural teeth missing side by side or for restructuring a completely new set of teeth without any remaining natural teeth.

DE 100 19 339 A1 and DE 103 33 013 A1 describe implant systems in which a medical element is each secured according to a method of the kind mentioned above.

The clipping connection according to the known methods or in the known medical elements must meet high requirements: On the one hand, it must be so easy to use and detach that the implant which has tightly grown together with the bone is not excessively loaded in the longitudinal direction in respect of tension and pressure. On the other hand, the force required for detaching the clipping connection must remain constant over a prolonged period of time, which is typically years and decades for substitute teeth, under high and changing loads between implant and medical element.

In the case of insufficient dimensioning of the clipping connection, the force required for detaching the connection can decrease by material fatigue to such an extent that the medical element will detach inadvertently from the implant.

It is the object of the invention to secure a medical element of the kind mentioned above against inadvertent detachment from the implant.

### SOLUTION

Based on the known methods, it is proposed in accordance with the invention that the wing is subsequently secured against radial movement by hardening of a curable compound in the receiving recess (9). The wing is thus radially movable without any limits in this way prior to the introduction of the securing element and can accordingly be inserted with a minimum expenditure of force into the implant. The wing can even be linked to the clip element in such a way that it does not produce any counterforce during the insertion into the implant. The clip element is fixed and secured in the desired position only by a firmly bonded connection with the cured mass. The wing is then blocked against radial movement to the inside and thus reliably and permanently prevents any inadvertent release of the medical element from the implant.

In a preferred embodiment of the method in accordance with the invention, the curable compound is cured in the receiving recess. Working with compounds curable under UV irradiation belongs to standard techniques in dentistry and is thus also very easily applicable in this context. Moreover, there are numerous curable compounds available at low prices which allow an individual adjustment of the method in accordance with the invention to each individual case.

Preferably, the wing is additionally secured against radial movement by means of a securing element introduced into the clip element within the scope of the method in accordance with the invention. The use of such a securing element not only increases the durability of the securing of the medical element in the implant, it also allows, without any curable compound, providing a preliminary securing of the medical element in the implant, e.g. in laboratory operations.

Within the scope of the method in accordance with the invention, the securing element is preferably introduced into a lead-through opening in the clip element, which opening starts out from the proximal end of the connecting pin. Such a continuous lead-through opening offers various options for arranging the method in accordance with the invention and thus increases its variability.

One end of a fiber-optic light element can be introduced in particular in the lead-through opening (14, 19, 27), which element will cure the curable compound by sending UV light through the fiber-optic light element and thereafter the end of the fiber-optic light element is severed, with the severed end of the fiber-optic light element remaining in the clip element as a securing element. By introducing a fiber-optic light element directly into a cavity of the clip element, curing is simplified with respect to manipulation and considerably accelerated through irradiation from the outside of the bone. Finally, the method is considerably simplified in such a way that the end of the fiber-optic light element remains in the clip element after curing. Complex monitoring of the process in order to prevent a gluing of the fiber-optic element can be omitted because this effect is even desirable in order to increase the stability of the connection.

In a further preferred embodiment of the method in accordance with the invention, the securing element is provided with an external thread and screwed into a respective internal thread of the lead-through opening in the clip element and/or fixed by means of an elastic securing ring in the longitudinal direction to an incision which is radially circumferential in the lead-through opening. The merely mechanical securing of the medical element simplifies manipulation on the one hand. On the other hand, the connections made are insensitive to chemical ageing processes in comparison with polymerizates. Manipulation of the implant system is further simplified by using a securing ring because the end position of the securing element is clearly defined in a tactile manner during installation in the medical element in accordance with the invention.

On the other hand, a method in accordance with the invention can be arranged in such a way that the securing element is introduced in a releasable fashion into the clip element. For example, a pin-like securing element can be pushed in a custom-fit manner into a cavity of the clip element in such a way that a radial movement of the movable wing and thus any inadvertent detachment of a medical element (e.g. a sealing cap, an impression post or a gingival former) which is inserted only temporarily into the implant is avoided reliably.

When such a curable compound is chosen within the scope of a method in accordance with the invention which can be depolymerised by using a solvent for example, the detachment of the medical element from the implant is further simplified.

In a preferred embodiment of the method in accordance with the invention, the implant with a medical element receiving therein is implanted in a bone. The medical element received during the implantation in the implant can be a manipulation element or a sealing or healing cap which is secured against inadvertent detachment by the method as described above. Manipulation of the implant system is thus clearly simplified. The receiving recess of the implant is reliably protected from contamination by implantation with inserted healing cap.

According to a method in accordance with the invention, the implant can be received as a model in an apparatus instead of a bone for adjusting the medical element in the laboratory. For example, an apparatus emulating the jawbone facilitates the adaptation in the laboratory in connection with the adjacent teeth. Receiving the implant in a holding or manipulation element facilitates comprehensive manual machining of the medical element, which is metal cutting, dyeing or applying layers of further material.

In such an apparatus, the securing element can be introduced into the clip element starting from the free end of the connecting pin. A short securing element which can be slid “from below” into the connecting pin can be integrated in the apparatus and be connected with a security switch in such a way that metal cutting of the medical element by means of electrically driven tools is only possible after securing has been made.

Within the scope of the method in accordance with the invention, the medical element can be connected with a second medical element via a web, with the web being fixed to the medical element in accordance with the invention by means of the securing element. The web can be fixed to the medical element in accordance with the invention by a circumferential collar or by individual, radially protruding noses on the securing element or by gluing with the securing element.

Based on the known medical elements it is proposed that the medical element can be secured by the corporeal features as described above in the implant in accordance with one of the methods mentioned above.

### EMBODIMENTS

The invention is now explained in closer detail by reference to embodiments, wherein:

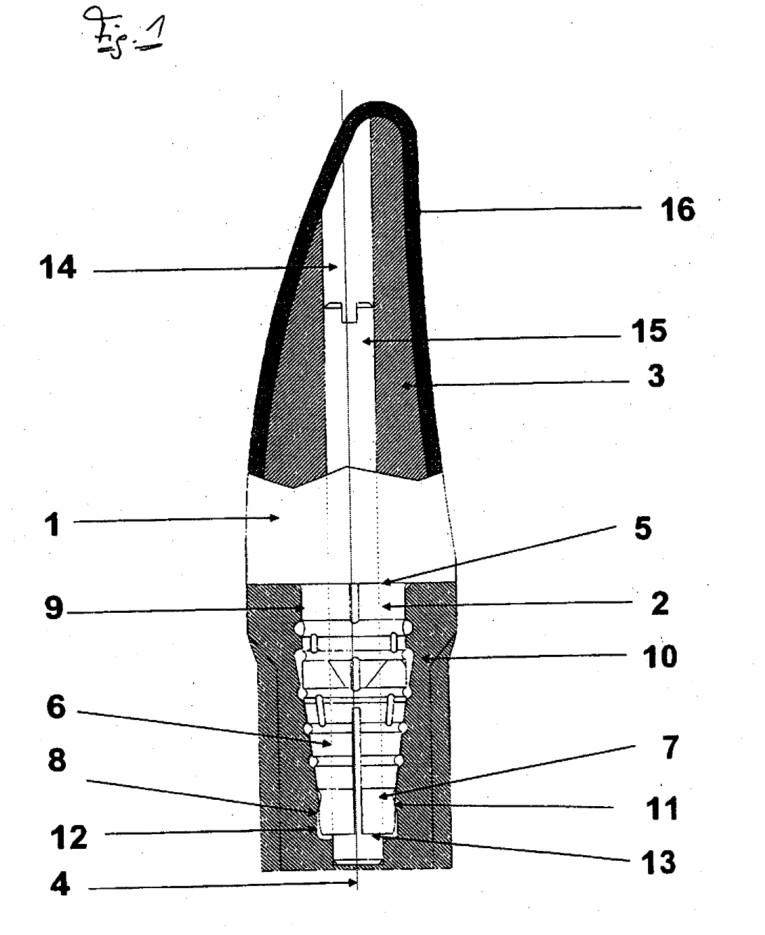

FIG. 1 shows a first medical element in accordance with the invention;

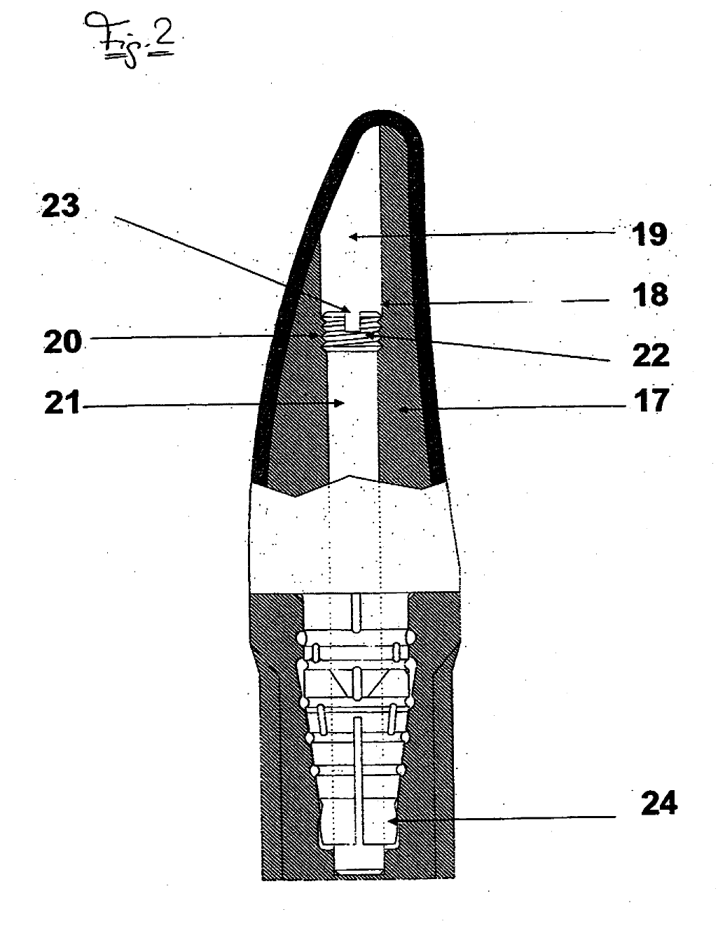

FIG. 2 shows a second medical element in accordance with the invention;

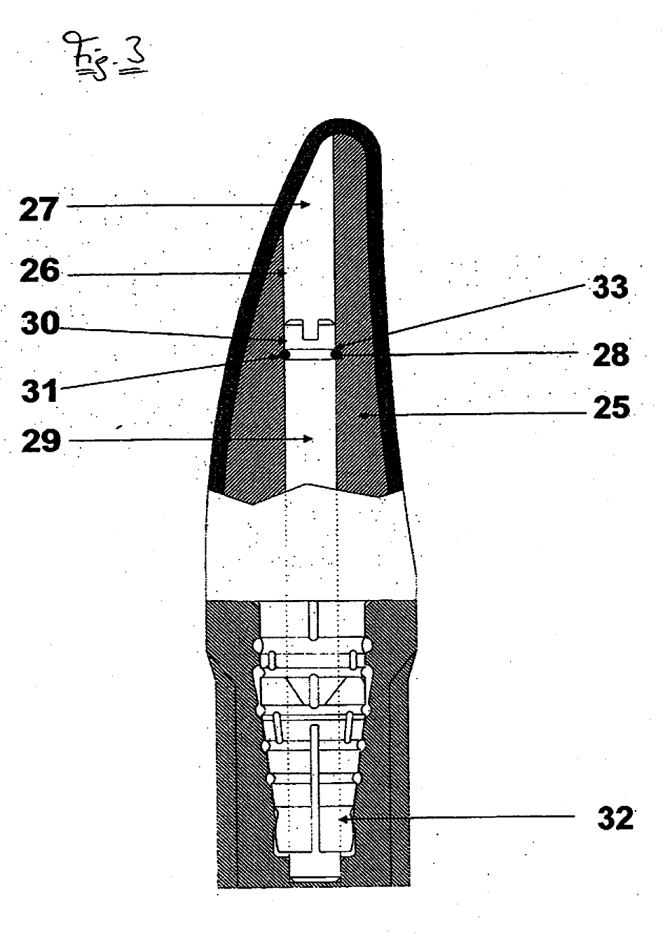

FIG. 3 shows a third medical element in accordance with the invention;

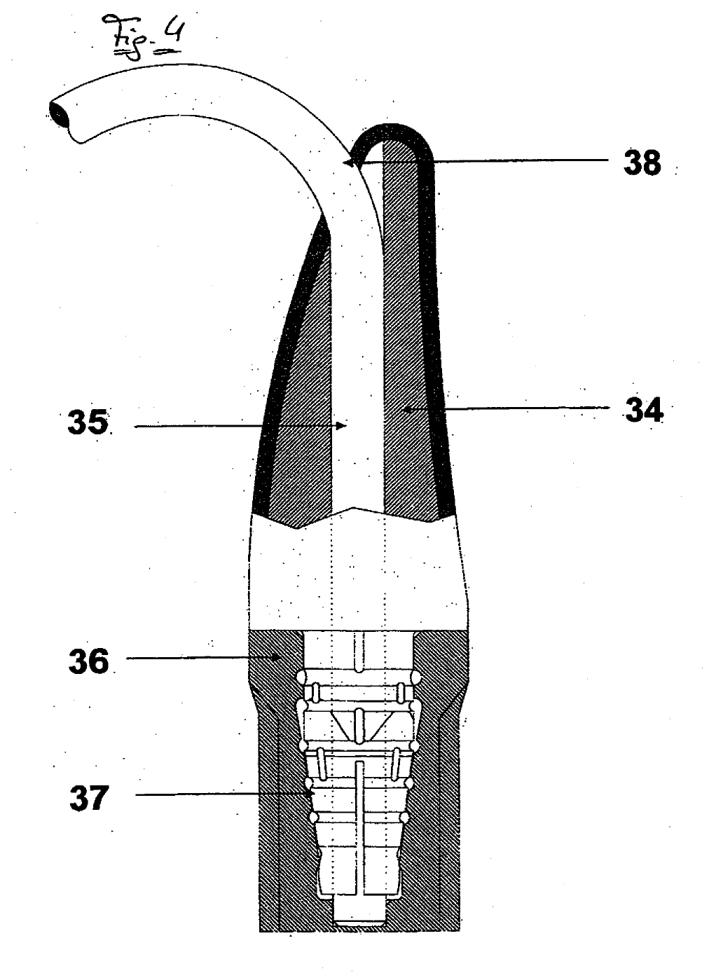

FIG. 4 shows a fourth medical element in accordance with the invention;

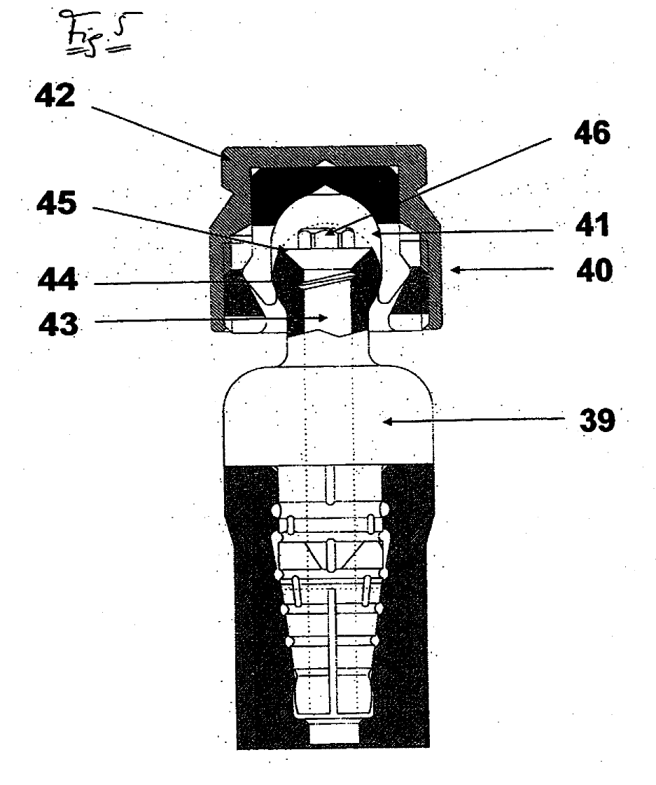

FIG. 5 shows a fifth medical element in accordance with the invention;

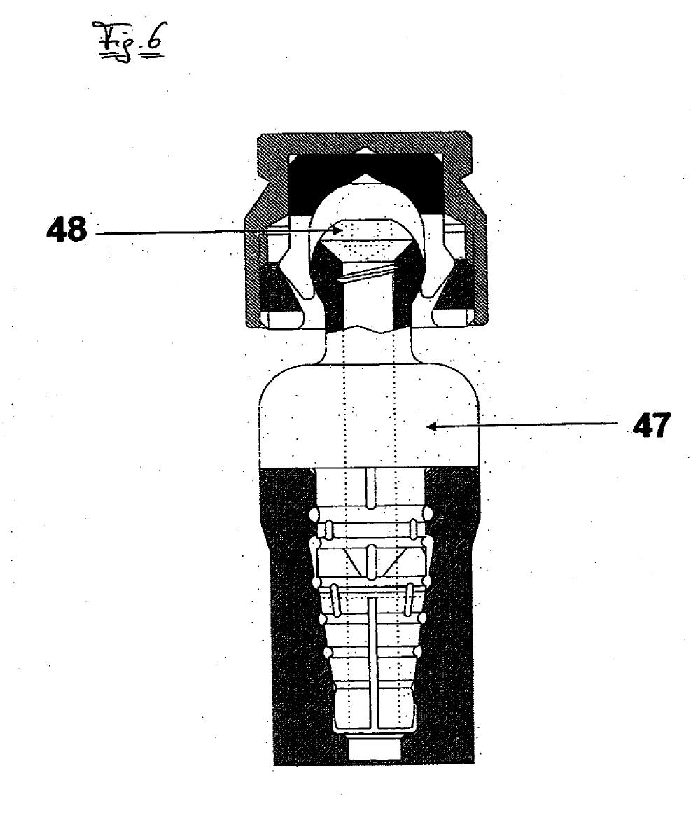

FIG. 6 shows a sixth medical element in accordance with the invention;

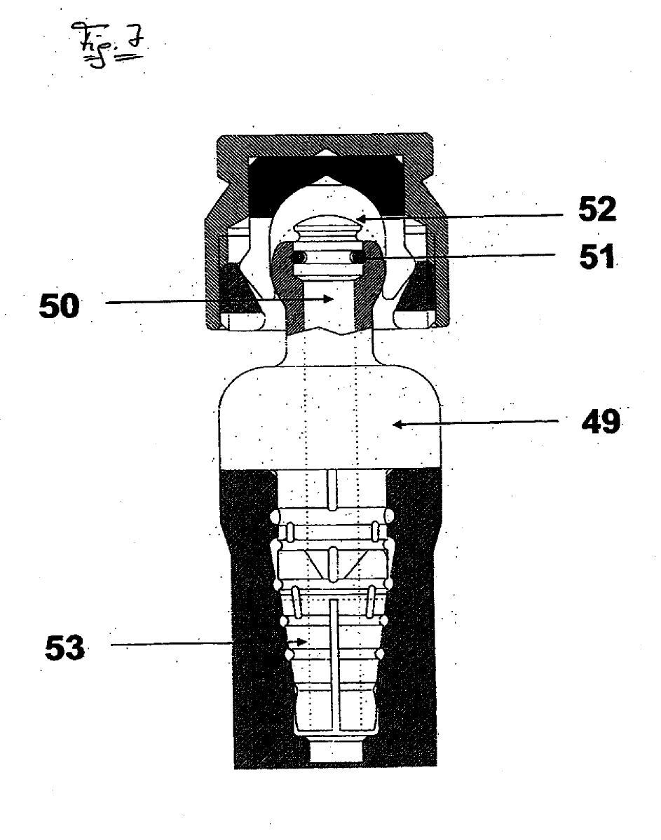

FIG. 7 shows a seventh medical element in accordance with the invention;

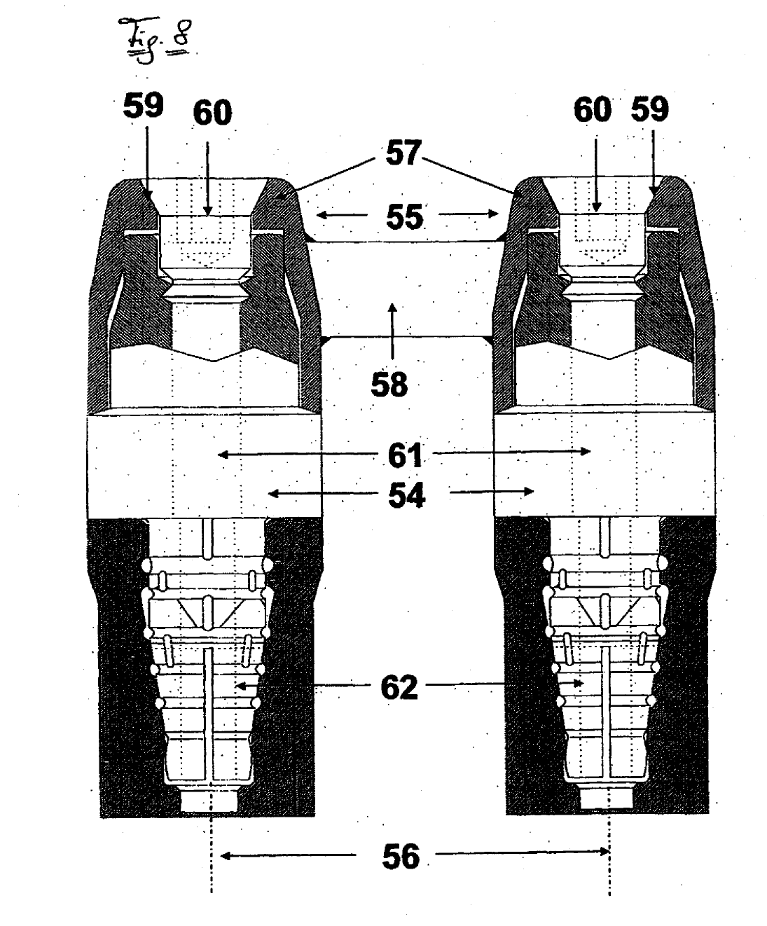

FIG. 8 shows an eighth medical element in accordance with the invention;

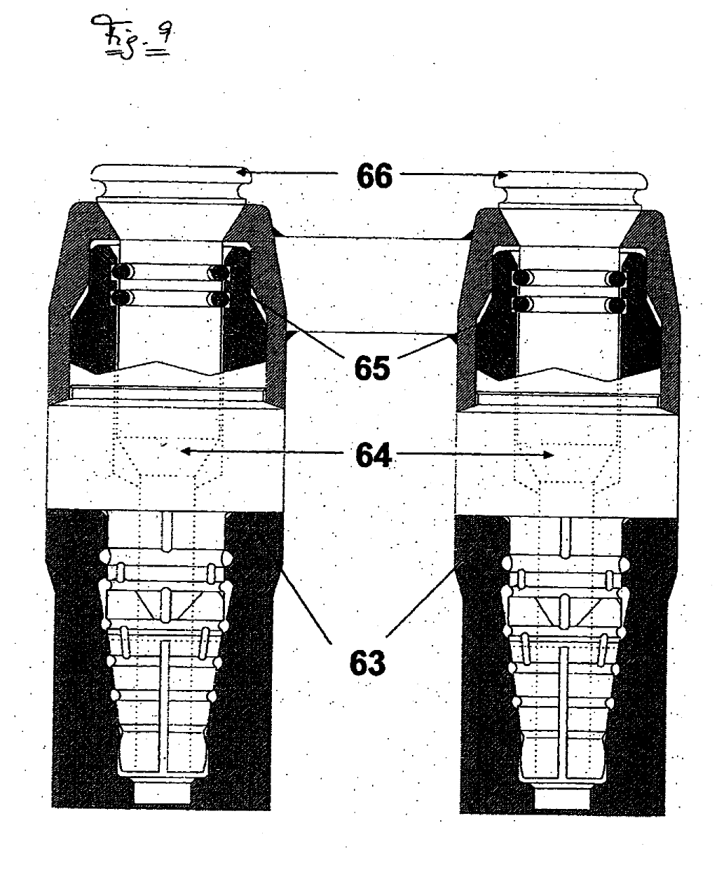

FIG. 9 shows a ninth medical element in accordance with the invention;

The first medical element 1 in accordance with the invention as shown in FIG. 1 comprises a connecting pin 2 and a supraconstruction 3 made of titanium. The connecting pin 2 is at first cylindrical in the longitudinal direction 4 of the connecting pin 2 in the direction of its distal end 5 and is then provided with a slightly conical tapering configuration. The connecting pin 2 comprises a clip element 6 with four wings 7 which comprise a radially outwardly protruding projection 8 and are movable transversally to the longitudinal direction 4 as a result of material elasticity.

The connecting pin 2 of the first medical element 1 is received in accordance with FIG. 1 in a receiving recess 9 of an implant 10 which can be implanted in a receiving bore in a bone (not shown). The receiving recess 9 extends in the longitudinal direction 4 into the implant 10 and is provided in its inner jacket surface 11 with a radially outwardly facing depression 12 which corresponds to the projection 8 on the wings 7.

The first medical element 1 comprises a lead-through opening 14 starting from its proximal end 13. A pin-like securing element 15 which prevents a radially inwardly directed movement of the wings 7 is introduced in an interlocking manner into the clip element 6 through the lead-through opening 14 in accordance with FIG. 1 and is guided through the same. The supraconstruction 3 of the first medical element 1 emulates the shape of an incisor and is provided on the surface with a ceramic layer 16. In order to form the crown of a substitute tooth, plastic is applied in layers onto the supraconstruction 3 in the laboratory, with the lead-through opening 14 being sealed with the plastic.

In contrast to the first medical element 1 as shown in FIG. 1, the second medical element 17 shown in FIG. 2 comprises a right-handed internal thread 20 in a section of the jacket surface 18 of the lead-through opening 19. The securing element 21 comprises on its head 22 a matching outside thread and a nose 23. For securing the clip element 24 of the second medical element 17, the securing element 21 is introduced into the lead-through opening 19, as in the first medical element 1, and finally screwed in with a few twists. The nose 23 on the securing element 21 is shown simplified in a slit-like manner in FIG. 2. In order to avoid damage to the internal thread 20, it will rather be arranged in practice so as to lie on the inside, e.g. as a hexagon. By loosening the screwed joint, the securing element 21 can be detached in an especially simple way from the second medical element 17.

In contrast to the second medical element 17 as shown in FIG. 2, the third medical element 25 as shown in FIG. 3 comprises a circumferential radial incision 28 in a section of jacket surface 26 of the lead-through opening 27. The securing element 29 also comprises in its jacket surface 30 a circumferential radial incision 31. For securing the clip element 32 of the third medical element 25, the securing element 29 is again introduced at first into the lead-through opening 27 until a securing ring 33 which rests in the lead-through opening 27 latches into the incision 31 on the securing element 29.

In contrast to the first medical element 1 as shown in FIG. 1, the securing element 35 is arranged as a fiber-optic light element in the fourth medical element 34 as shown in FIG. 4. In order to secure the fourth medical element 34, a plastic filling material which can be cured under UV-light and which is not shown here (as in the other embodiment) is brought between the implant 36 and the medical element 34, the securing element 35 is introduced into the clip element 37 and subjected to UV-light. After curing the filling material it is severed in a manner not shown at the distal end 38 of the medical element 34.

The fifth medical element 39 as shown in FIG. 5 differs from the second medical element 17 as shown in FIG. 2 substantially by the supraconstruction 40. It comprises a ball head 41 on which dentures which are merely indicated here in a principal sectional view or a partial prosthesis (not shown) can be fixed in a releasable manner according to the “press button” principle instead of by means of a denture fixative. The securing element 43 is secured by means of a thread 44 in the fifth medical element 39, which thread is only indicated here. The nose 45 on the securing element 43 is arranged as a hexagon with a conical seat 46 in the supraconstruction 40.

The sixth medical element 47 as shown in FIG. 6 differs from the fifth medical element 39 shown in FIG. 5 only by the nose 48 which is arranged here as an inside hexagon.

The seventh medical element 49 shown in FIG. 7 substantially differs from the fifth medical element 39 shown in FIG. 5 in such a way that the securing element 50, as in the third medical element 25 according to FIG. 3, is latched into the medical element by means of the securing ring 51. The head 52 of the securing element 50 is additionally slightly undercut in a mushroom-like manner, so that the securing element 50 can be detached easily from the medical element 49 and the clip element 53 can thus be released again.

The similar eighth medical elements 54 as shown in FIG. 8 differ from the sixth medical element 47 as shown in FIG. 6 by the outer shape of the supraconstruction 55 which is arranged in a substantially cylindrical way in the longitudinal direction 56 of the medical elements 54. In the illustration in accordance with FIG. 8, two caps 57 are placed on the medical elements 54, which caps are fixedly connected on their part by a web 58. The caps 57 are fixed by a conical seat 59 on head 60 of the securing elements 61 to the medical elements 54. A prosthesis (not shown) “rides” on said web 58, as a result of which mechanical stress and wear and tear of the clip elements 62 are reduced considerably.

The similar ninth medical elements 63 as shown in FIG. 9 differ from the eighth medical elements 54 in accordance with FIG. 8 by the manner of fixing the securing elements 64. As in the seventh medical element 49 in accordance with FIG. 7, the securing elements 64 are secured by securing rings 65, of which there are two each, in the ninth medical elements 63. As in the seventh medical element 49, the head 66 of the securing elements 64 is undercut in a mushroom-like manner in order to ensure easy releasing capability.

The reference numerals in the Figs. are as follows:
1 Medical element
2 Connecting pin
3 Supraconstruction
4 Longitudinal direction
5 Distal end
6 Clip element
7 Wing
8 Projection
9 Receiving recess
10 Implant
11 Jacket surface
12 Depression
13 Proximal end
14 Lead-through opening
15 Securing element
16 Ceramic layer
17 Medical element
18 Jacket surface
19 Lead-through opening
20 Internal thread
21 Securing element
22 Head
23 Nose
24 Clip element
25 Medical element
26 Jacket surface
27 Lead-through opening
28 Incision
29 Securing element
30 Jacket surface
31 Incision
32 Clip element
33 Securing ring
34 Medical element
35 Securing element
36 Implant
37 Clip element
38 Distal end
39 Medical element (34)
40 Supraconstruction
41 Ball head
42 Denture
43 Securing element
44 Thread
45 Nose
46 Conical seat
47 Medical element
48 Nose
49 Medical element
50 Securing element
51 Securing ring
52 Head
53 Clip element
54 Medical element
55 Supraconstruction
56 Longitudinal direction
57 Cap
58 Web
59 Conical seat
60 Head
61 Securing element
62 Clip element
63 Medical element
64 Securing element
65 Securing ring
66 Head

1. (canceled)

2. The method according to claim 14, wherein the curable compound is cured in the receiving recess by UV-light.

3. The method according to claim 14 wherein the wing is additionally secured against radial movement by means of a securing element introduced into the clip element.

4. The method according to claim 3, wherein the securing element is introduced into the clip element in a lead-through opening, which opening starts out from a proximal end of the connecting pin.

5. The method according to claim 2 wherein one end of a fiber-optic light element is introduced into the lead-through opening, and wherein the curable mass is cured by injecting UV-light into the fiber-optic light element, whereupon the end of the fiber-optic light element is severed and the severed end of the fiber-optic light element is left in the clip element as a securing element.

6. The method according to claim 5, wherein the securing element is provided with an external thread and screwed into a respective internal thread of the lead-through opening in the clip element.

7. The method according to claim 6, wherein the securing element is fixed by means of an elastic securing ring in the longitudinal direction to an incision which is radially circumferential in the lead-through opening.

8. The method according to claim 7, wherein the securing element is detachably introduced into the clip element.

9. The method according to claim 14, wherein the implant is implanted in a bone with the medical element received therein.

10. The method according to claim 14, wherein the implant is received in the manner of a model in an apparatus for adaptation of the medical element in the laboratory.

11. The method according to claim 10, wherein the securing element is introduced into the clip element starting from the free end of the connecting pin.

12. The method according to claim 14, wherein the medical element is connected with a second medical element via a web, and the web is fixed to the medical element by means of a securing element.

13. (canceled)

14. A method for securing a medical element in an implant comprising the steps of:
receiving a transversally movable connecting pin of the medical element relative to and extending in longitudinal direction into an interior of the implant, engaging a projection on at least one wing of a clip element of the connecting pin in a radially outwardly facing depression in a jacket surface of a receiving recess in the implant, providing a curable compound in the receiving recess and subsequently securing the wing against radial movement by hardening of the curable compound in the receiving recess.

15. A medical element comprising a connecting pin received in a receiving recess of an implant, said receiving recess extending in a longitudinal direction into an interior of the implant which is implantable in a receiving bore in a bone, wherein the connecting pin has a clip element provided with at least one wing that is movable transversely with respect to the longitudinal direction, said wing has a radially outwardly protruding projection for engagement with a corresponding depression in a jacket surface of the receiving recess, and wherein the medical element is secured in the implant by fixing the wing against radial movement through the hardening of a curable compound present in the receiving recess.

16. The medical element according to claim 15, wherein the curable compound is cured in the receiving recess by UV-light.

17. The medical element according to claim 15, wherein the wing is additionally secured against radial movement by means of a securing element introduced into the clip element.

18. The medical element according to claim 17, wherein the securing element is introduced into the clip element in a lead-through opening, which opening starts out from the proximal end of the connecting pin.

19. The medical element according to claim 18, wherein one end of a fiber-optic light element introduced into the lead-through opening, and wherein the curable mass is cured by injecting UV-light into the fiber-optic light element, whereupon the end of the fiber-optic light element is severed and the severed end of the fiber-optic light element is left in the clip element as a securing element.

20. The medical element according to claim 18, wherein the securing element is provided with an external thread and screwed into a respective internal thread of the lead-through opening in the clip element.

21. The medical element according to claim 20, wherein the securing element is fixed by means of an elastic securing ring in the longitudinal direction to an incision which is radially circumferential in the lead-through opening.

22. The medical element according to claim 17, wherein the securing element is detachably introduced into the clip element.

23. The medical element according to claim 22, wherein securing element is introduced into the clip element starting from a free end of the connecting pin.

24. The medical element according to claim 15, wherein the medical element is connected with a second medical element via a web, and the web is fixed to the medical element by means of a securing element.
